(19) United States
(12) Patent Application Publication
(10) Pub. No.: US 2018/0271516 A1
(43) Pub. Date: Sep. 27, 2018

(54) SYSTEM AND METHOD FOR STAY SUTURE MANAGEMENT

(71) Applicant: LSI Solutions, Inc., Victor, NY (US)

(72) Inventor: Jude S. Sauer, Pittsford, NY (US)

(21) Appl. No.: 15/933,228

(22) Filed: Mar. 22, 2018

Related U.S. Application Data
(60) Provisional application No. 62/475,106, filed on Mar. 22, 2017.

(51) Int. Cl. A61B 17/04; A61B 17/062; A61B 17/06

(52) U.S. Cl. CPC A61B 17/0469; A61B 17/0482; A61B 17/0485; A61B 17/06066; A61B 17/062

(57) ABSTRACT

A system for stay suture management is disclosed. The system has a catheter and a hooking manipulator. The hooking manipulator has a distal tip configured as an obturator to help the catheter push through muscle tissue while preventing tissue from entering the catheter. The hooking manipulator also has a hook configured to enter the proximal opening of the catheter, pass all the way through the catheter, and exit a distal opening of the catheter. The hooking manipulator further has a handle. Another system for stay suture management is disclosed, having a catheter and a tube passing through the catheter and extending from both a proximal opening and a distal opening of the catheter. A snare loop extends from the tube extending from the distal opening of the catheter. A handle is coupled to the snare loop on the side of the tube extending from the proximal opening of the catheter.

### FIELD

The claimed invention relates to surgical devices, and more specifically to a system and method for stay suture management.

### BACKGROUND

Laparoscopic, endoscopic, and other types of minimally invasive surgical procedures often rely on percutaneous introduction of surgical instruments into an internal region of a patient where the surgical procedure is to be performed. As part of many minimally invasive surgical procedures, stay sutures may be placed in various tissue and then tensioned either to pull the tissue out of the way or to move the tissue to a more convenient position for the surgeon to reach through a minimally invasive incision. Surgeons continue to find it desirable to utilize smaller and smaller access incisions in order to minimize trauma and reduce patient recovery times. In some situations, the minimally invasive access incision may only allow the surgeon to either set up the stay sutures or perform the ensuing surgical procedure, but does not allow for easy adjustment of the stay sutures once the ensuing surgical procedure is underway. Therefore, it would be desirable to have an improved device for routing the stay sutures separately from a main surgical access point while enabling convenient adjustment of the stay suture tensions.

### SUMMARY

A system for stay suture management is disclosed. The system has a catheter and a hooking manipulator. The hooking manipulator has a distal tip configured as an obturator to help the catheter push through muscle tissue while preventing tissue from entering the catheter. The hooking manipulator also has a hook configured to enter the proximal opening of the catheter, pass all the way through the catheter, and exit a distal opening of the catheter. The hooking manipulator further has a handle.

Another system for stay suture management is disclosed. The system has a catheter. The system also has a tube passing through the catheter, the tube extending from both a proximal opening and a distal opening of the catheter. The system further has a snare loop extending from the tube extending from the distal opening of the catheter. The system also has a handle coupled to the snare loop on the side of the tube extending from the proximal opening of the catheter.

A further system for stay suture management is disclosed. The system has a tube having an proximal opening and a distal opening. The system also has a snare loop extending from the distal opening of the tube. The system further has a handle coupled to the snare loop on the proximal opening side of the tube.

### BRIEF DESCRIPTION OF THE DRAWINGS

FIG. 1A illustrates one embodiment of a system for stay suture management.

FIG. 1B illustrates another embodiment of a system for stay suture management with the addition of a target for holding a snare loop.

FIG. 1C illustrates a further embodiment of a system for stay suture management which is pre-packaged as inserted through a catheter device.

FIG. 2A is a top view illustrating one embodiment of a hooking manipulator for use with a catheter device.

FIG. 2B illustrates an embodiment of a system for stay suture management which utilizes the hooking manipulator of FIG. 2A, shown in a side view.

FIG. 3A illustrates one embodiment of a catheter device and a corresponding obturator which is removed from the catheter device.

FIG. 3B illustrates the obturator of FIG. 3A inserted into the catheter device of FIG. 3A.

FIG. 4A is a top view illustrating another embodiment of a hooking manipulator for use with a catheter device.

FIG. 4B illustrates an embodiment of a system for stay suture management which utilizes the hooking manipulator of FIG. 4A, shown in a side view.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

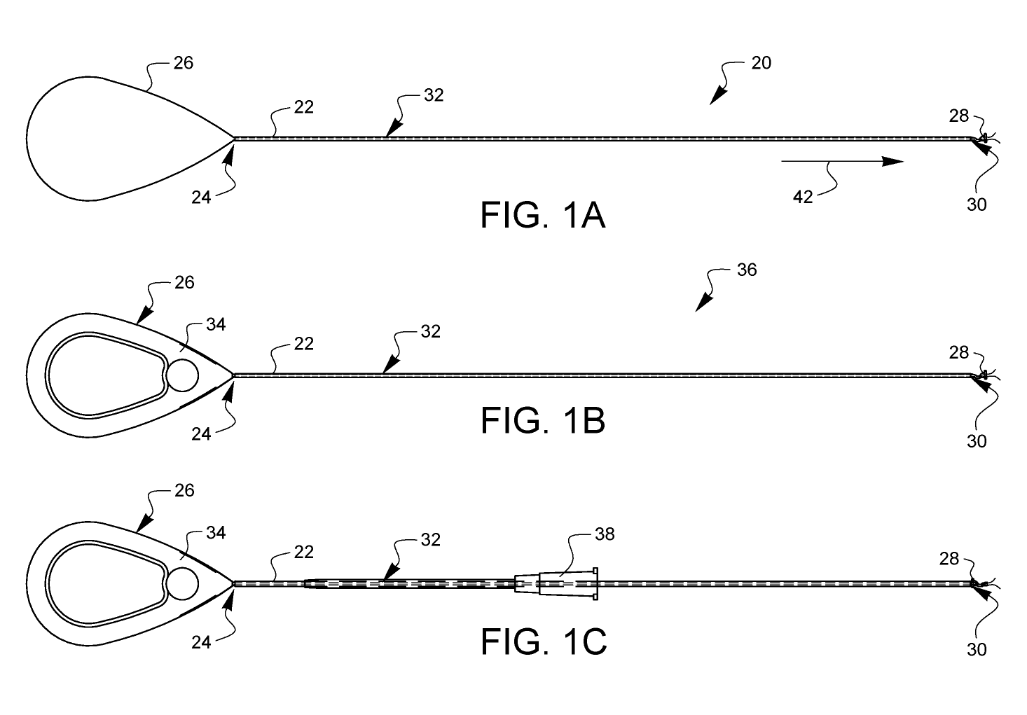

FIG. 1A illustrates one embodiment of a system 20 for stay suture management.

The system 20 has a tube 22 having a distal opening 24 from which a snare loop 26 protrudes. The snare loop 26 is coupled to a handle 28 which protrudes from a proximal opening 30 of the tube. The snare loop 26 is coupled to the handle 28 by at least one middle snare component 32 which passes through the tube 22. In the embodiment of FIG. 1A, the snare loop 26 is formed of a suture, for example, but not limited to a polypropylene 3-0 suture whose ends have been fed back through the tube 22 and mechanically held together by a mechanical “knot” or fastener acting as the handle 28. One suitable type of mechanical knot is the Ti-KNOT® titanium fastener or the COR-KNOT® titanium fastener available from LSI Solutions, Inc. in Victor, N.Y. (See www.lsisolutions.com). In such an embodiment, the two ends of the suture which reside in the tube are the middle snare component coupling the handle 28 to the snare loop 26.

In order to keep the loop 26 from prematurely being pulled back through the tube 22, a target 34 may be placed within the loop 26, such as with the system 36 of FIG. 1B. All other features of the system 36 are similar to those of the FIG. 1A. The target 34 may be removed when it is desired to use the snare loop 26.

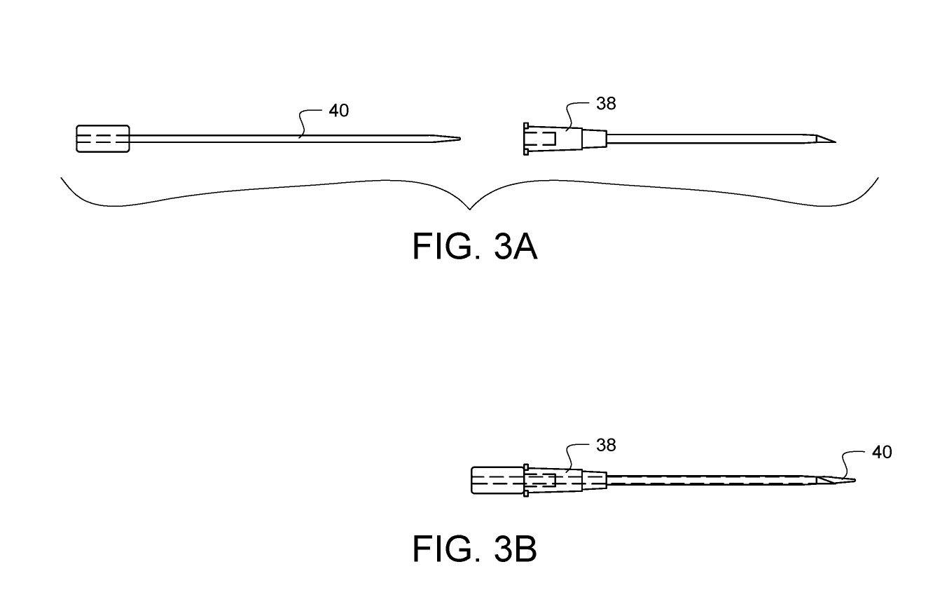

Before discussing the use of the snare systems of FIGS. 1A and 1B, it may be helpful to consider a surgical situation and the intravenous catheter 38 and obturator 40 embodiments of FIGS. 3A and 3B. In modern minimally invasive surgery, especially minimally invasive cardiac surgery, surgeons are often able to perform a heart valve repair or replacement procedure through a very small incision in-between a patient's ribs without the need for a sternotomy or a rib dissection. While such an incision provides adequate access to a surgical field, it does not lend itself to providing access angles on which the surgeon may pull stay sutures away from the surgical field in order to properly expose the tissue being worked on. For example, when replacing an aortic valve, it is helpful to be able to place stay sutures at the commissure points of the existing valve and also within the wall of the aorta in order to maintain an opening of an aortotomy which provides access to the valve in the first place. A small catheter device 38, similar to an intravenous catheter, may be used to pierce through the skin and muscle tissue at locations aside from and peripheral to the minimally invasive incision. With an obturator 40, enough rigidity may be provided to reach the thoracic cavity, and then the obturator may be removed, leaving an extremely small catheter in place. Meanwhile, the surgeon can place a desired stay suture by stitching it into tissue via the minimally invasive incision. A suture management system, such as the system 20 of FIG. 1A may also be used effectively at this point. The handle 28 may be pulled proximally 42 until the loop 26 just disappears back into the distal opening 24 of the tube 22. The tube 22 is preferably sized to pass through the intravenous catheter 38 with the obturator 40 removed. FIG. 1C illustrates one way the catheter 38 could be pre-packaged with the system 20. To access the catheter 38, the target 34 would need to be removed so that the catheter 38 could be slid off the end. The catheter 38 could be placed in the patient using an obturator as described above (or be stiff enough not to need an obturator). As noted above, with the loop 26 pulled back into the tube 22, the distal end of the tube 22 may be passed through the catheter into the thoracic cavity of the patient. Once thoracic access is gained, the snare loop 26 may be pushed back out of the distal opening. The proximal end of the snare tube 22 may be manipulated externally so that the distal end of the snare loop 26 is moved into approximation with the stay suture that has been placed (or will be placed, depending on the order of events). The stay suture can be placed through the snare loop 26, and then the handle 28 can be pulled so that the stay suture exits the proximal end 30 of the tube 22. Depending on the embodiment and surgeon preference, the intravenous catheter may be modified to have a suture tie-down on which the stay suture may be tensioned. In other embodiments, the tube 22 may remain in the patient and the used as a Rumel-Tourniquet whereby the proximal end of the suture tube is clamped to hold the stay suture. The stay sutures exiting the catheter may also be clamped, held, or otherwise tensioned in a variety of ways known to those skilled in the art.

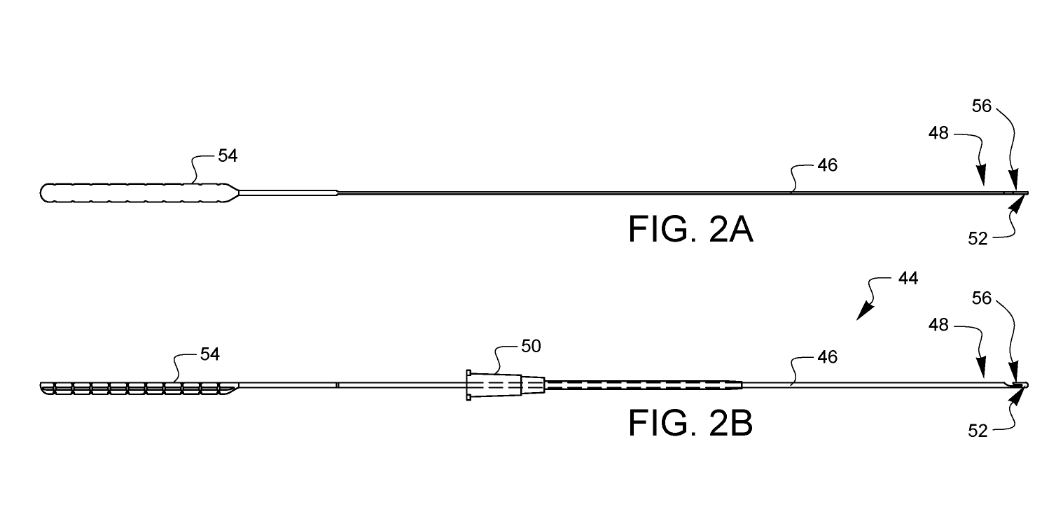

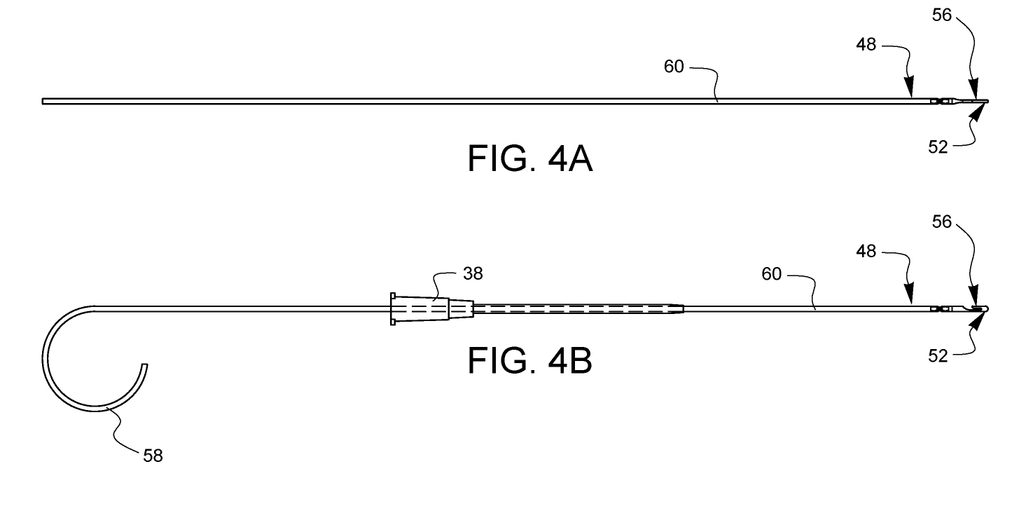

Alternately, a hooking manipulator may be provided to pass through the intracatheter. FIG. 2A illustrates one embodiment of a hooking manipulator 46 for such use from a top view. The hooking manipulator 46 has a distal end 48 which is sized to fit through a small intracatheter-sized catheter. The distal end has a hook 56 which is not easily visible in the top view. The hooking manipulator 46 also has a handle 54, the flat of which is facing upward in the top view of FIG. 2A to align with the hook 56. The flat on the handle may be considered an orientation feature which corresponds with the hook. Other embodiments may have other types of orientation features, such as, but not limited to labeling or different texturing. FIG. 2B illustrates one embodiment of a system 44 for stay suture management which incorporates the hooking manipulator 46. In FIG. 2B, the hooking manipulator 46 is shown in a side view. The distal end 48 of the hooking manipulator 46 is sized to fit through a small intracatheter-sized catheter 50. In FIG. 2B, the catheter 50 is shown installed on the hooking manipulator 46. Depending on the embodiment, the hooking manipulator 46 could have a very-distal end 52 which acts as an obturator when the hooking manipulator 46 is pulled back into the catheter 50. Other embodiments may provide a separate obturator as with the previous devices. Once the catheter 50 has been placed through the skin and tissue and has reached the thoracic cavity, the handle 54 of the hooking manipulator 46 may be used to move the distal end 48 of the manipulator 46 into approximation with the stay suture that has been placed (or will be placed, depending on the order of events). The stay suture can be grabbed by the hook 56 and the handle 54 pulled so that the stay suture exits the proximal end of the catheter 50. The stay suture then may be clamped, held, or tensioned as desired. Some embodiments may also be provided with a tube that passes through the catheter 50 (the hooking manipulator being sized to pass through the tube in such embodiments) if a Rumel-Tourniquet setup is desired. FIG. 4B illustrates a similar embodiment to that of FIG. 2B, however, the hooking manipulator 60 has a different style handle 58 which is curved for ease of holding by placing a finger therein. FIG. 4B is from a side view, while FIG. 4A illustrates just the hooking manipulator 60 from a top view.

Various advantages of a system and method for stay suture management have been discussed above. In particular, these systems for stay suture management provide off-axis stay suture orientation that is typically only possible with open heart surgery without the needs for a full sternotomy. This improves visualization during surgery and most importantly, it enables quicker patient recovery times and less post-operative pain. The extremely small catheters may be removed with minimal bleeding and may not even require suturing. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A system for stay suture management, comprising:
a) a catheter; and
b) a hooking manipulator having:
1) a distal tip configured as an obturator to help the catheter push through muscle tissue while preventing tissue from entering the catheter;
2) a hook configured to enter the proximal opening of the catheter, pass all the way through the catheter, and exit a distal opening of the catheter; and
3) a handle.

2. The system of claim 1, wherein the handle has an orientation feature which corresponds to the hook.

3. The system of claim 2, wherein the orientation feature comprises a flat on the handle.

4. The system of claim 1, wherein the handle comprises a textured surface.

5. The system of claim 1, wherein the handle comprises a curved handle configured for placement of a finger therein.

6. The system of claim 1, wherein the hooking manipulator is installed in the catheter.

7. A system for stay suture management, comprising:
a) a catheter;
b) a tube passing through the catheter, the tube extending from both a proximal opening and a distal opening of the catheter;
c) a snare loop extending from the tube extending from the distal opening of the catheter; and
d) a handle coupled to the snare loop on the side of the tube extending from the proximal opening of the catheter.

8. The system of claim 7, wherein the snare loop comprises suture.

9. The system of claim 7, wherein the handle comprises a mechanical fastener.

10. The system of claim 7, further comprising a target within the snare loop on the side of the tube extending from the distal opening of the catheter.

11. The system of claim 7, further comprising an obturator.

12. A system for stay suture management, comprising:
a tube having an proximal opening and a distal opening;
a snare loop extending from the distal opening of the tube; and
a handle coupled to the snare loop on the proximal opening side of the tube.

13. The system of claim 12, wherein the snare loop comprises a suture.

14. The system of claim 12, wherein the handle comprises a mechanical fastener.

15. The system of claim 12, further comprising a target within the snare loop on the distal opening side of the tube.

16. The system of claim 12, further comprising a catheter.

17. The system of claim 16, further comprising an obturator.
